(19) United States
(12) Patent Application Publication
(10) Pub. No.: US 2010/0143887 A1
(43) Pub. Date: Jun. 10, 2010

(54) BIOSENSOR AND METHOD FOR DETECTING BIOMOLECULES BY USING THE BIOSENSOR

(75) Inventors: Ansoon KIM, Daejeon (KR); Chil Seong AH, Daejeon (KR); Chan Woo PARK, Daejeon (KR); Jong-Heon YANG, Daejeon (KR); Chang-Geun AHN, Daejeon (KR); Taeyoub KIM, Seoul (KR); In Bok BAEK, Cheongju-si (KR); Wanjoong KIM, Goyang-si (KR); Gun Yong SUNG, Daejeon (KR); Seon Hee PARK, Daejeon (KR)

Correspondence Address: LAHIVE & COCKFIELD, LLP;FLOOR 30, SUITE 3000, ONE POST OFFICE SQUARE, BOSTON, MA, 02109, US

(73) Assignee: Electronics and Telecommunications Research Institute, Daejeon (KR)

(21) Appl. No.: 12/551,996

(22) Filed: Sep. 1, 2009

(30) Foreign Application Priority Data
Dec. 5, 2008 (KR) 10-2008-0123237
Mar. 31, 2009 (KR) 10-2009-0027383

(51) Int. Cl. C12Q 1/70; C12Q 1/68; G01N 33/53

(52) U.S. Cl. 435/5; 435/6; 435/7.1

(57) ABSTRACT

Provided are a biosensor and a method for detecting biomolecules by using the biosensor. The biosensor includes a detection unit and a fluid channel. The detection unit is disposed on a substrate and has a surface to which detection target molecules binding specifically to probe molecules are immobilized. The fluid channel is configured to provide an analysis solution containing the probe molecules to the detection target molecules. The probe molecules bind specifically to the target molecules and the detection target molecules.

### CROSS-REFERENCE TO RELATED APPLICATIONS

This U.S. non-provisional patent application claims priority under 35 U.S.C. §119 of Korean Patent Application Nos. 10-2008-0123237, filed on Dec. 5, 2008, and 10-2009-0027383, filed on Mar. 31, 2009, the entire contents of which are hereby incorporated by reference.

### BACKGROUND OF THE INVENTION

The present invention disclosed herein relates to a biosensor and a method for detecting biomolecules by using the biosensor, and more particularly, to a biosensor capable of detecting less-charged or non-charged biomolecules electrochemically and a method for detecting biomolecules by using the biosensor.

Biosensors are devices that can selectively detect an analysis target material by converting a biological interaction or reaction into an electrical or optical signal by means of an electrical or optical transducer and a biological acceptor reacting on a specific material. The biosensors are widely applied in the technical field of measuring the concentration of biochemical materials that are clinically valuable. Examples of the biosensor application field include medicine (clinical diagnosis), drug manufacture, environment, food, military, and research. The characteristics of the biosensor industries vary depending on their application fields. The demand for biosensors is greatest in the medical field, and medical biosensors are expected to lead the development of the biosensor industries.

Electrochemical biosensors, which electrochemically detect a reaction between an enzyme and a biochemical material, are being used most widely among various biosensors. The electrochemical biosensors are very useful because they can convert the amount of biological sample into an electrical signal that is easy to process. In particular, a field-effect transistor (FET) biosensor detects biomaterials, which are macromolecules charged and adsorbed to the biosensor, by measuring a current that varies according to an electric field change caused by the biomaterials. Among the devices detecting target materials by means of electrical signals, a transistor-based biosensor can be fabricated through a conventional semiconductor process. Therefore, the transistor-based biosensor can be integrated and miniaturized, thus making it possible to reduce the costs.

### SUMMARY OF THE INVENTION

The present invention provides a biosensor capable of detecting less-charged or non-charged biomolecules electrochemically.

The present invention also provides a method for detecting less-charged or non-charged biomolecules electrochemically.

The objects of the present invention are not limited to the aforesaid, and other objects not described herein will be clearly understood by those skilled in the art from descriptions below.

Embodiments of the present invention provide biosensors for detecting target molecules, including: a detection unit disposed on a substrate and having a surface to which detection target molecules binding specifically to probe molecules are immobilized; and a fluid channel configured to provide an analysis solution containing the probe molecules to the detection target molecules, wherein the probe molecules bind specifically to the target molecules and the detection target molecules.

In other embodiments of the present invention, methods for detecting biomolecules include: preparing an analysis solution containing conjugates of analysis target molecules and probe molecules, and remainder probe molecules; supplying the analysis solution to a fluid channel of a biosensor including: a detection unit disposed on a substrate and having a surface to which detection target molecules binding specifically to probe molecules are immobilized; and the fluid channel configured to provide the analysis solution containing the probe molecules binding specifically to the detection target molecules and the analysis target molecules to the detection target molecules; binding the remainder probe molecules to the detection target molecules of the detection unit; and measuring an electrical conductance change in the detection unit.

The details of other embodiments are included in the detailed description and the drawings.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is a perspective view of a biosensor according to an exemplary embodiment of the present invention;

FIG. 2 is a cross-sectional view of the biosensor according to an exemplary embodiment of the present invention;

FIG. 3 is a flow diagram illustrating a biomolecule detection method according to an exemplary embodiment of the present invention;

FIGS. 4 to 6 are diagrams illustrating a biomolecule detection method according to an exemplary embodiment of the present invention;

FIG. 7 is a diagram illustrating a method for detecting target molecules from an analysis solution containing high-concentration target molecules according to an exemplary embodiment of the present invention;

FIG. 8 is a diagram illustrating a method for detecting target molecules from an analysis solution containing low-concentration target molecules according to an exemplary embodiment of the present invention; and

FIG. 9 is a graph illustrating an electrical conductance change of a biosensor depending on the concentration of target molecules.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art. Like reference numerals refer to like elements throughout the specification.

In the following description, the technical terms are used only for explaining specific exemplary embodiments while not limiting the present invention. The terms of a singular form may include plural forms unless otherwise specified. The meaning of “include,” “comprise,” “including,” or “comprising,” specifies a property, a region, a fixed number, a step, a process, an element and/or a component but does not exclude other properties, regions, fixed numbers, steps, processes, elements and/or components. It will also be understood that when a layer (or film) is referred to as being “on” another layer or substrate, it can be directly on the other layer or substrate, or intervening layers may also be present.

Additionally, the embodiments in the detailed description will be described with reference to sectional views or plan views as ideal exemplary views of the present invention. In the drawings, the dimensions of layers and regions are exaggerated for clarity of illustration. Accordingly, shapes of the exemplary views may be modified according to manufacturing techniques and/or allowable errors. Therefore, the embodiments of the present invention are not limited to the specific shape illustrated in the exemplary views, but may include other shapes that may be created according to manufacturing processes. Areas exemplified in the drawings have general properties, and are used to illustrate specific shapes of device regions. Thus, these should not be construed as limiting to the scope of the present invention.

In the specification, target molecules are biomolecules with specific natures, which may be interpreted as having the same meaning as assays or analytes. In exemplary embodiments of the present invention, the biomolecules correspond to antigens.

In the specification, probe molecules are biomolecules binding specifically to target molecules, which may be interpreted as having the same meaning as receptors or acceptors. In exemplary embodiments of the present invention, the probe molecules correspond to antibodies.

When a solution containing target molecules flows into a detection unit to which probe molecules are immobilized, the probe molecules of the detection unit bind specifically to the target molecules and an electrochemical biosensor detects the target molecules by detecting an electrical conductance change according to the charge quantity of the target molecules. Specifically, when the probe molecules and the target molecules bind specifically in the detection unit, the amount of a current flowing in a channel changes due to a change in the surface charge transferred into the channel. Then the electrochemical biosensor measures the current of the channel to detect the target molecules. Because the biosensor detects the surface charge transferred by the target molecules to the channel, the target molecules must be charged and the detection performance increases with an increase in the charge quantity.

The biosensor must be able to detect the target molecules even when the target molecules are non-charged (i.e., electrically neutral) or less-charted and small in molecular weight.

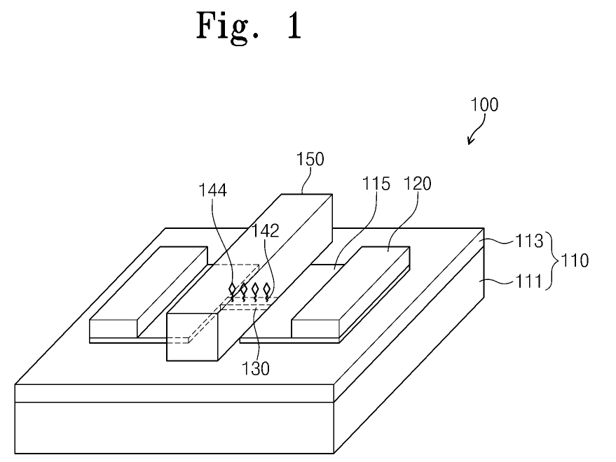

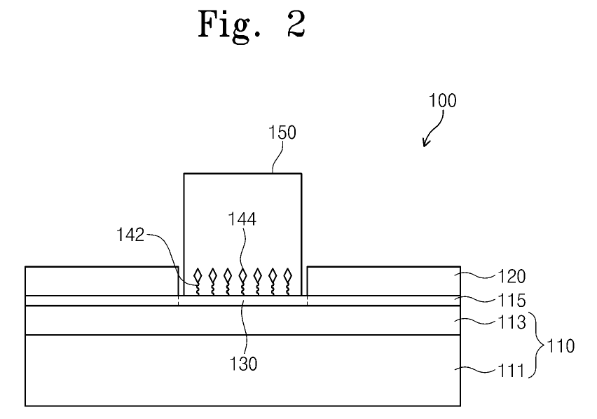

Hereinafter, a biosensor according to an exemplary embodiment of the present invention will be described in detail with reference to FIGS. 1 and 2.

FIG. 1 is a perspective view of a biosensor according to an exemplary embodiment of the present invention. FIG. 2 is a cross-sectional view of the biosensor according to an exemplary embodiment of the present invention.

Referring to FIGS. 1 and 2, a biosensor 100 according to an exemplary embodiment of the present invention includes a substrate 110, source/drain electrodes 120, a detection unit 130, detection target molecules 144, and a fluid channel 150.

The substrate 110 may be a bulk semiconductor substrate, a glass substrate, or a plastic substrate. Also, the substrate 110 may be a substrate formed of dielectric material such as titanium oxide, acrylic resin, epoxy resin and polyimide resin. Also, a silicon-on-insulator (SOI) substrate may be used as the substrate 110 in order to reduce the leakage current of the biosensor and increase the driving current. In an exemplary embodiment of the present invention, an SOI substrate is exemplified as the substrate 110. The SOI substrate 110 may include: a support substrate 111 for mechanical support; an insulating layer 113 on the support substrate 111; and a doped layer 115 on the insulating layer 113.

The insulating layer 113 may be formed of oxide material or nitride material in order to prevent an electrical short between the support substrate 111 and the doped layer 115. For example, the insulating layer 113 may be formed of silicon oxide or silicon nitride. Examples of the silicon oxide are High Density Plasma (HDP), Boron Phosphorus Silicate Glass (BPSG), Phosphorus Silicate Glass (PSG), Plasma Enhanced Tetra Ethyle Ortho Silicate (PETEOS), Undoped Silicate Glass (USG), Fluorinated Silicate Glass (FSG), Carbon Doped Oxide (CDO), and Organo Silicate Glass (OSG).

The doped layer 115 may be an impurity region formed by diffusion of n-type or p-type impurities in the support substrate 111, or an ion implanted layer formed by implantation of impurity ions, or an epitaxial layer formed by epitaxial growth.

The source/drain electrodes 120 are disposed on the substrate 110. The source/drain electrodes 120 are spaced apart from each other by a predetermined distance, and a voltage may be applied to the source/drain electrodes 120. The detection unit 130, i.e., a channel is disposed between the source/drain electrodes 120. The doped layer 115 may be disposed under the source/drain electrodes 120 to electrically connect the detection unit 130 and the source/drain electrodes 120. In another exemplary embodiment, the source/drain electrodes 120 may be impurity regions in the semiconductor substrate 110 doped with impurities.

The channel between the source/drain electrodes 120 is the detection unit 130 detecting biomolecules, and may be formed of material whose electrical characteristic change by an external electric field. For example, the channel may include crystalline silicon, amorphous silicon, a doped layer, a semiconductor layer, an oxide layer, a compound layer, a carbon nano tube (CNT), or a semiconductor nanowire. In exemplary embodiments of the present invention, the detection unit 130 includes a doped layer. The detection unit 130 including a doped layer may be formed to a nano size in order to improve the sensitivity of the biosensor 100.

Detection target molecules 144 are immobilized to the surface of the detection unit 130 in order to detect non-charged biomolecules or less-charged biomolecules of small molecular weight. The detection target molecules 144 may be immobilized to the surface of the detection unit 130 directly or using linkers 142 as an intermediate medium. The detection target molecules 144 immobilized to the detection unit 130 may be biomolecules that have specific natures and bind specifically to probe molecules. For example, the detection target molecules 144 may be protein, nucleic acid, organic molecules, inorganic molecules, oxide, or metal oxide. The protein molecule may be any biomolecule such as antigen, antibody, matrix protein, enzyme, and coenzyme. The nucleic acid may be DNA, RNA, PNA, LNA, or a mixture thereof.

The detection target molecules 144 may be immobilized to the surface of the detection unit 130 by physisorption, chemical adsorption, covalent binding, electrostatic attraction, copolymerization, or avidin-biotin affinity system.

The surface of the detection unit 130 may be surface-treated to induce the linkers 142 to immobilize the detection target molecules 144 more tightly. Specifically, a functional group may be induced on the surface of the detection unit 130 in order to immobilize the detection target molecules 144 to the surface of the detection unit 130. Examples of the functional group immobilized to the surface of the detection unit are isothiol group, carbonyl group, carboxyl group, amine group, imine group, epoxy group, nitro group, hydroxyl group, pheny group, nitrile group, isocyano group, isothiocyno group, thiol group, and silane group.

The fluid channel 150 disposed across the detection unit 130, and analysis target biomolecules may be provided through the fluid channel 150 to the surface of the detection unit 130. That is, the fluid channel 150 serves to provide an analysis solution containing biomolecules to the detection unit 130. The analysis solution contains analysis target molecules, probe molecules, and nonspecific molecules. The analysis solution may be physiological body fluid such as blood, plasma, serum, interstitial fluid, lavage, perspiration, saliva, and urine.

In an exemplary embodiment of the present invention, the analysis target molecules in the analysis solution include non-charged molecules or less-charged molecules of small molecular weight. The probe molecules specifically bind selectively to detection/analysis target molecules. Also, the probe molecules have a greater charge quantity than those of the analysis target molecules. For example, the probe molecules may be protein, cell, virus, nucleic acid, organic molecules, or inorganic molecules. The protein may be any biomaterial such as antigen, antibody, matrix protein, enzyme, and coenzyme. The nucleic acid may be DNA, RNA, PNA, LNA, or a mixture thereof.

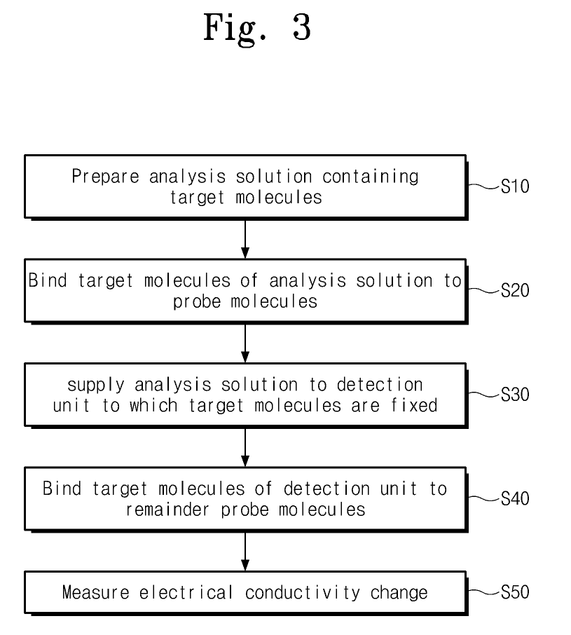

Hereinafter, a biomolecule detection method according to an exemplary embodiment of the present invention will be described in detail with reference to FIGS. 3 to 8.

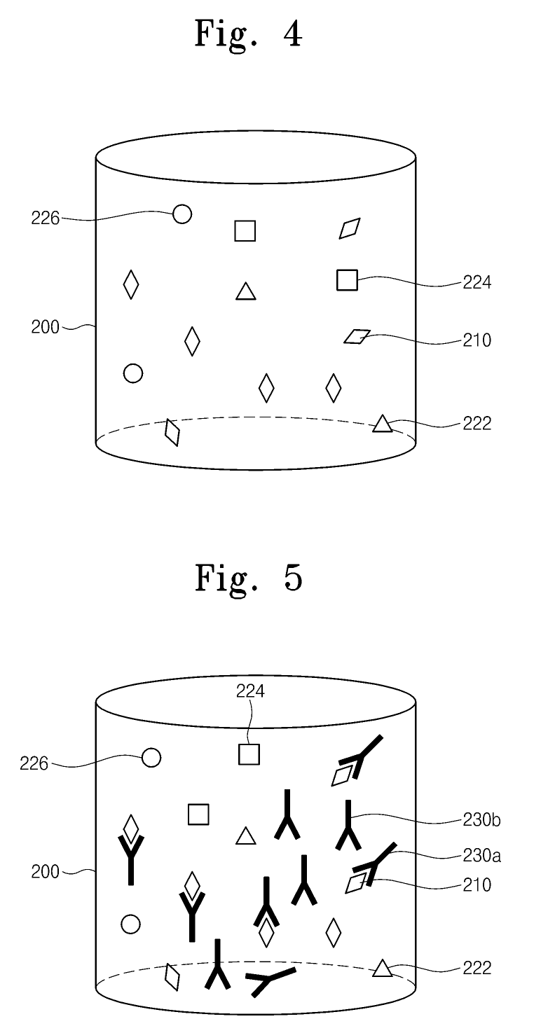

FIG. 3 is a flow diagram illustrating a biomolecule detection method according to an exemplary embodiment of the present invention. FIG. 4 is a diagram illustrating an analysis solution containing analysis target molecules according to an exemplary embodiment of the present invention. FIG. 5 is a diagram illustrating an analysis solution containing analysis target molecules and probe molecules according to an exemplary embodiment of the present invention. FIG. 6 illustrates providing an analysis solution to the biosensor according to an exemplary embodiment of the present invention.

Referring to FIGS. 3 and 4, an analysis solution 200 containing non-charged analysis target molecules 210 or less-charged analysis target molecules 210 of small molecular weight a prepared (S10).

The analysis solution 200 may be physiological body fluid such as blood, plasma, serum, interstitial fluid, lavage, perspiration, saliva, and urine. The analysis solution 200 contains non-charged analysis target molecules 210 or less-charged analysis target molecules 210 of small molecular weight, which have specific natures and bind specifically to the probe molecules. Also, the analysis solution 200 may contain nonspecific molecules 222, 224 and 226 that do not bind to the probe molecules.

The analysis target molecules 210 in the analysis solution 200 may be nucleic acid, cell, virus, protein, organic molecules, or inorganic molecules. The protein molecule may be any biomaterial such as antigen, antibody, matrix protein, enzyme, and coenzyme. The nucleic acid may be DNA, RNA, PNA, LNA, or a mixture thereof.

Referring to FIGS. 3 and 5, probe molecules 230a and 230b are bound to the analysis target molecules 210 in the analysis solution 200 (S20).

That is, the probe molecules 230a and 230b are provided into the analysis solution 200 containing the non-charged target molecules 210 or the less-charged analysis target molecules 210 of small molecular weight. When the probe molecules 230a and 230b are provided into the analysis solution 200, target molecule 210/probe molecule 230a conjugates are created.

The probe molecules 230a and 230b do not bind to the nonspecific molecules 222, 224 and 226 but bind specifically to the analysis target molecules 210. The probe molecules 230a and 230b are charged so much as to detect an electrical conductance change in the detection unit 130 of the biosensor (see FIG. 1).

The concentration of the probe molecules 230a and 230b in the analysis solution 200 is higher than the concentration of the analysis target molecules 210 so that all the analysis target molecules 210 in the analysis solution 200 can bind to the probe molecules 230a and 230b. Accordingly, some (e.g., the probe molecules 230a) of the probe molecules bind to the analysis target molecules 210, and the others (e.g., the probe molecules 230b) remain in the analysis solution 200 without binding to the analysis target molecules 210. That is, almost all the analysis target molecules 210 in the analysis solution 200 bind to the probe molecules 230a.

Referring to FIGS. 3 and 6, the analysis solution 200 is provided to the detection unit 130 of the biosensor, which has the detection target molecules 144 immobilized thereto (S30).

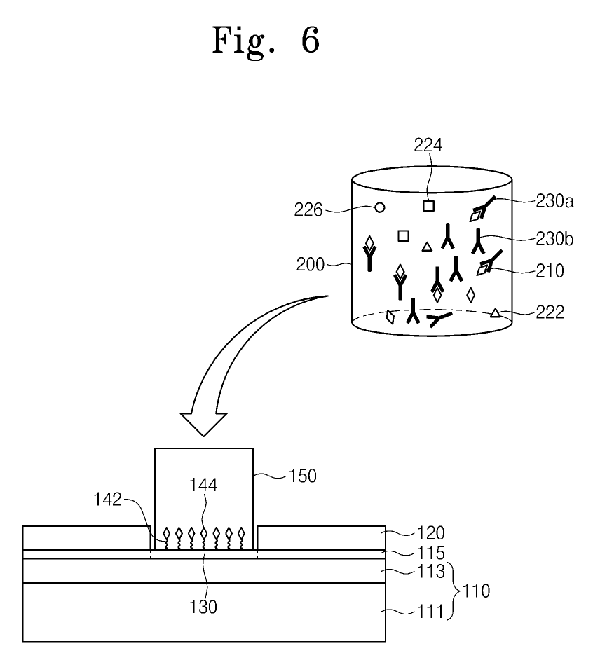

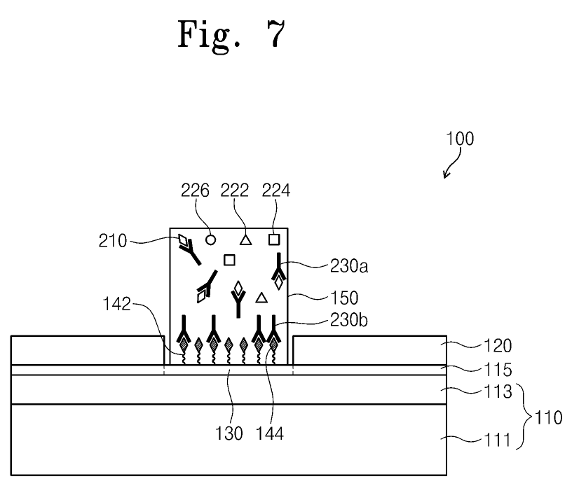

That is, the analysis solution 200 containing the target molecule 210/probe molecule 230a conjugates and the remainder probe molecules 230b is provided to the detection unit 130 that has the detection target molecules 144 immobilized thereto. Accordingly, the remainder probe molecules 230b in the analysis solution 200 bind specifically to the detection target molecules 144 of the detection unit 130 (S40). The amount of the remainder probe molecules 230b binding specifically to the detection target molecules 144 of the detection unit 130 may vary depending on the concentration of the analysis target molecules 210 in the analysis solution 200. This will be described later in detail with reference to FIGS. 7 and 8.

Thereafter, an electrical conductance change in the detection unit 130, in which the detection target molecules 144 and the remainder probe molecules 230b bound together, is measured (S50). That is, because the probe molecules charged so much as to detect an electrical conductance change bind to the detection target molecules 144 of the detection unit 130, a current flows in the detection unit 130 when a voltage is applied to the source/drain electrodes 120. Thus, a change in the current flowing in the detection unit 130 (i.e., the channel) is measured according to a change in the surface charge density of the probe molecules 230b immobilized to the surface of the detection unit 130.

FIG. 7 is a diagram illustrating a method for detecting analysis target molecules from an analysis solution containing high-concentration target molecules according to an exemplary embodiment of the present invention.

Referring to FIG. 7, the biosensor 100 may have an n-type doped layer 115. Because a large amount of non-charged or less-charged analysis target molecules 210 are present in a high-concentration analysis solution 200a, there are a small number of remainder probe molecules 230b that do not bind to the analysis target molecules 210 of the analysis solution 200a. Accordingly, there are a small number of remainder probe molecules 230b that are immobilized to the surface of the detection unit 130 by binding specifically to the detection target molecules 144 of the detection unit 130.

That is, in the case of the high-concentration analysis solution 200a, the quantity of a surface charge transferable to the detection unit 130 decreases due to a decrease in the number of the remainder probe molecules 230b that are immobilized on the detection unit 130 and have a negative charge. Accordingly, an electrical conductance change in the detection unit 130 decreases when a voltage is applied to the source/drain electrodes 120.

FIG. 8 is a diagram illustrating a method for detecting analysis target molecules from an analysis solution containing low-concentration target molecules according to an exemplary embodiment of the present invention.

Referring to FIG. 8, the biosensor 100 may have an n-type doped layer 115. Because a small amount of non-charged or less-charged analysis target molecules 210 are present in a low-concentration analysis solution 200b, there are a large number of remainder probe molecules 230b that do not bind to the analysis target molecules 210 of the analysis solution 200a. Accordingly, there are a large number of remainder probe molecules 230b that are immobilized to the surface of the detection unit 130 by binding specifically to the detection target molecules 144 of the detection unit 130.

That is, in the case of the low-concentration analysis solution 200b, the quantity of a surface charge transferred to the detection unit 130 increases due to an increase in the number of the remainder probe molecules 230b that are immobilized to the surface of the detection unit 130 and have a negative charge. Accordingly, an electrical conductance change in the detection unit 130 increases when a voltage is applied to the source/drain electrodes 120.

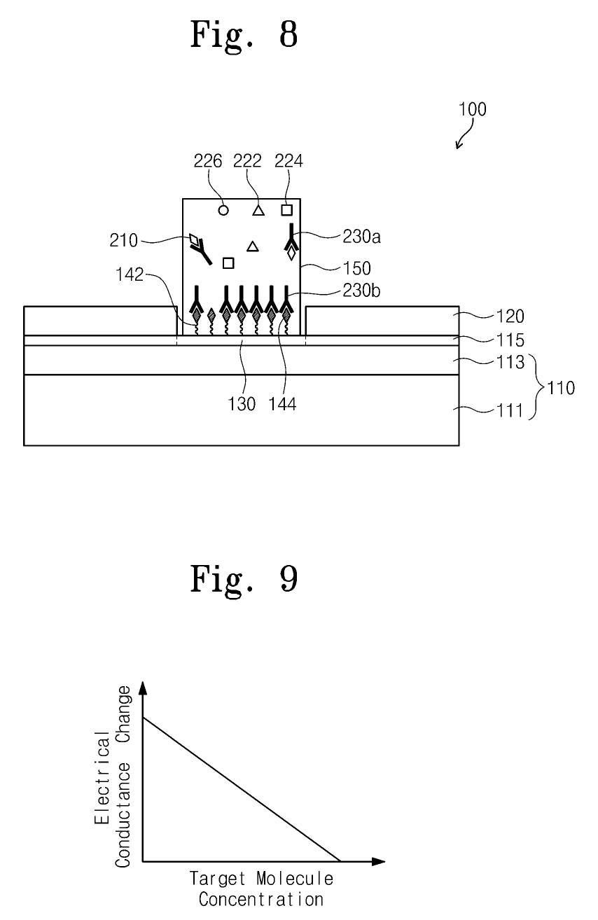

FIG. 9 is a graph illustrating an electrical conductance change depending on the concentration of target molecules, which is obtained using reference analysis solutions with known analysis target molecule concentrations. That is, FIG. 9 illustrates an electrical conductance change depending on the concentration of target molecules, which is caused by probe molecules that remain without binding specifically in the analysis solution. As illustrated in FIG. 9, the electrical conductance change is inversely proportional to the concentration of the analysis target molecules in the analysis solution. Accordingly, the concentration of the analysis target molecules in the analysis solution can be quantized by measuring the electrical conductance change caused by the remainder probe molecules in the analysis solution.

As described above, the biosensor and the biomolecule detection method using the biosensor according to the present invention can detect non-charged target molecules or less-charged target molecules of small molecular weight electrochemically. That is, the present invention can detect target molecules of small charge quantity.

That is, the present invention binds the less-charged target molecules in the analysis solution to the probe molecules and binds the remaining probe molecules to the detection target molecules of the biosensor, thereby detecting the target molecules in the analysis solution.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A biosensor for detecting target molecules, comprising:
a detection unit disposed on a substrate and having a surface to which detection target molecules binding specifically to probe molecules are immobilized; and
a fluid channel configured to provide an analysis solution containing the probe molecules to the detection target molecules,
wherein the probe molecules bind specifically to the target molecules and the detection target molecules.

2. The biosensor of claim 1, wherein the target molecules and the detection target molecules are the same biomaterial.

3. The biosensor of claim 1, wherein the probe molecules have a greater charge quantity than those of the target molecules and the detection target molecules.

4. The biosensor of claim 1, wherein the detection unit includes a semiconductor layer, a doped layer, an oxide layer, a compound layer, a carbon nano tube (CNT), or a semiconductor nanowire.

5. The biosensor of claim 1, further comprising source/drain electrodes connected electrically to both sides of the detection unit.

6. The biosensor of claim 5, further comprising an impurity region disposed under the source/drain electrodes and connected to the detection unit.

7. The biosensor of claim 1, wherein the substrate is one selected from the group consisting of a monocrystalline silicon substrate, a silicon-on-insulator (SOI) substrate, a glass substrate, and a plastic substrate.

8. The biosensor of claim 1, wherein the detection target molecules are immobilized to the surface of the detection unit by means of linkers induced on the surface of the detection unit.

9. The biosensor of claim 8, wherein the linkers is at least one selected from the group consisting of isothiol group, carbonyl group, carboxyl group, amine group, imine group, epoxy group, nitro group, hydroxyl group, pheny group, nitrile group, isocyano group, isothiocyno group, thiol group, and silane group.

10. A method for detecting biomolecules, comprising:
preparing an analysis solution containing conjugates of analysis target molecules and probe molecules, and remainder probe molecules;
supplying the analysis solution to a fluid channel of a biosensor including: a detection unit disposed on a substrate and having a surface to which detection target molecules binding specifically to probe molecules are immobilized; and the fluid channel configured to provide the analysis solution containing the probe molecules binding specifically to the detection target molecules and the analysis target molecules to the detection target molecules;
binding the remainder probe molecules to the detection target molecules of the detection unit; and
measuring an electrical conductance change in the detection unit.

11. The method of claim 10, wherein the probe molecules have a greater charge quantity than those of the target molecules and the detection target molecules.

12. The method of claim 10, wherein the analysis target molecules and the detection target molecules are the same biomaterial.

13. The method of claim 10, wherein the preparing of the analysis solution comprises:
preparing a solution containing the analysis target molecules;
providing probe molecules to the solution; and
binding the analysis target molecules to the probe molecules to form the conjugates.

14. The method of claim 13, wherein the concentration of the probe molecules in the solution is higher than the concentration of the analysis target molecules.

15. The method of claim 14, wherein the remainder probe molecules are probe molecules that are not bound to the analysis target molecules.

16. The method of claim 10, wherein the binding between the probe molecules and the analysis/detection target molecules include nucleic acid hybridization, antigen-antibody reaction, or enzyme-linked reaction.

17. The method of claim 10, wherein the analysis solution is one selected from the group consisting of blood, serum, plasma, interstitial fluid, lavage, perspiration, urine, and saliva.

18. The method of claim 10, wherein the probe molecules and the analysis/detection target molecules is at least one selected from the group consisting of nucleic acid, cell, virus, protein, organic molecules, or inorganic molecules.

19. The method of claim 18, wherein the nucleic acid is at least one selected from the group consisting of DNA, RNA, PNA, LNA, or a mixture thereof.

20. The method of claim 18, wherein the protein is at least one selected from the group consisting of include enzyme, matrix, antigen, antibody, ligand, aptamer, or receptor.
